(19) United States
(12) Patent Application Publication
(10) Pub. No.: US 2002/0179087 A1
(43) Pub. Date: Dec. 5, 2002

(54) Pharmaceutical compositions containing an oxitropium salt and a betamimetic

(76) Inventors: Karl-Heinz Bozung, Mainz (DE); Michel Pairet, Stromberg (DE); Richard Reichl, Gau-Algesheim (DE); Alexander Walland, Ingelheim (DE)

Correspondence Address: BOEHRINGER INGELHEIM CORPORATION, 900 RIDGEBURY ROAD, P. O. BOX 368, RIDGEFIELD, CT, 06877, US

(21) Appl. No.: 10/051,516

(22) Filed: Jan. 17, 2002

Related U.S. Application Data
(60) Provisional application No. 60/291,843, filed on May 17, 2001.

(30) Foreign Application Priority Data
Feb. 1, 2001 (DE) 101 04 370.8

(51) Int. Cl. A61M 11/00; A61M 16/10; B05D 7/14; B65D 83/06; A61K 9/14; A01N 59/00

(52) U.S. Cl. 128/203.15; 128/203.12; 128/200.14; 424/502; 424/723; 424/489

(57) ABSTRACT

Pharmaceutical composition comprising: (a) an oxitropium salt; and (b) a betamimetic, and their use in treating inflammatory or obstructive diseases of the respiratory tract in a patient in need of such treatment.

### RELATED APPLICATIONS

Benefit under 35 U.S.C. §119(e) of prior provisional application Ser. No. 60/291,843, filed May 16, 2001, is hereby claimed.

### FIELD OF THE INVENTION

The present invention relates to novel pharmaceutical compositions based on long-acting beta₂-agonists and oxitropium salts, processes for preparing them and their use in the treatment of respiratory complaints.

### BACKGROUND OF THE INVENTION

It is known from the prior art that betamimetics may successfully be used to treat obstructive diseases of the respiratory tract. The long-acting betamimetics salmeterol and formoterol are of particular importance. These compounds may be used effectively for treating chronic obstructive pulmonary disease (COPD) or asthma, for example.

However, administering these compounds to humans may be linked to undesirable side effects. The central side effects may be general malaise, excitement, sleeplessness, anxiety, trembling fingers, sweats and headaches. These side effects are not eliminated by inhaling the compounds even though they are generally then observed to a rather lesser extent than when the compounds are administered orally or parenterally. Of significantly greater importance in the use of the abovementioned compounds as antiasthmatic agents, for example, are the sometimes marked stimulant effects of these compounds on the heart. They lead to tachycardia, palpitations, angina pectoris-like pain and arrhythmias. Depending on the patient's physical constitution, these latter side effects on the heart may assume life-threatening proportions. It has now been observed that the side effects on the heart caused by salmeterol and formoterol may occur with worrying severity particularly at the start of the duration of activity of these pharmaceutical compositions. In therapy, salmeterol and formoterol are usually administered twice a day. Even if obstructive pulmonary diseases such as asthma or COPD are successfully treated by the administration of salmeterol or formoterol, the side effects described above must be expected to occur after every application of these two active substances.

The aim of the present invention is to provide drug combinations which will reduce the occurrence of the abovementioned side effects after the administration of long-acting betamimetics. A further aim of the present invention is to provide drug combinations by means of which the occurrence of the abovementioned side effects is reduced, particularly in the period shortly after the administration of the long-acting betamimetics. A further aim of the present invention is to provide drug combinations which will reduce the occurrence of the abovementioned side effects, particularly in the period shortly after the administration of the long-acting betamimetics, without counteracting the therapeutic effect intended to be achieved by the administration of the betamimetics.

A further aim of the present invention is to provide drug combinations which will reduce the occurrence of the abovementioned side effects after the administration of long-acting betamimetics which occurs several times a day, preferably twice a day. A further aim of the present invention is to provide drug combinations which will reduce the occurrence of the abovementioned side effects, particularly in the period shortly after the administration of long-acting betamimetics which occurs several times a day, preferably twice a day. A further aim of the present invention is to provide drug combinations which will reduce the occurrence of the abovementioned side effects, particularly in the period shortly after the administration of long-acting betamimetics which occurs several times a day, preferably twice a day, without counteracting the therapeutic effect which is being sought by the administration of the betamimetics.

### DESCRIPTION OF THE DRAWINGS

FIG. 1 shows an exploded view of the Handihaler® inhaler for administering the pharmaceutical combination according to the invention in inhalettes;

FIG. 2a shows a longitudinal section of the RESPIMAT® nebulizer disclosed in WO 97/12687 through the atomizer with the spring under tension; and

FIG. 2b shows a longitudinal section of the RESPIMAT® nebulizer disclosed in WO 97/12687 through the atomizer with the spring released.

FIGS. 2a and 2b herein are identical to FIGS. 6a and 6b of WO 97/12687.

### DETAILED DESCRIPTION OF THE INVENTION

Surprisingly, it has been found that the aims set out above can be achieved if one or more oxitropium salts 1 are used together with the long-acting betamimetics 2.

The compound oxitropium bromide, a salt of oxitropium, is known in the art. It is already used with great success as VENTILAT® for treating respiratory complaints, for example. Oxitropium salts 1 have the following chemical structure:

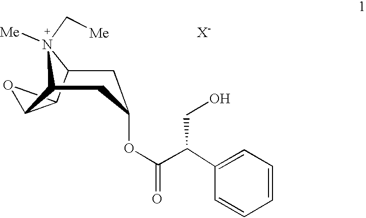

In the case of oxitropium bromide, X denotes bromine. This compound may also be referred to by its chemical name [7(S)-(1α,2β,4β,5α,7β)]-9-ethyl-7-(3-hydroxy-1-oxo-2-phenylpropoxy)-9-methyl-3-oxa-9-azoniatricyclo[3.3.1.0^{2,4}]nonane bromide. The name oxitropium for the purposes of the present invention is to be understood as a reference to the free cation 1′.

It has been found that oxitropium salts 1 are suitable for effectively counteracting the side effects, some of them extremely serious, caused by the betamimetics 2. It has also been found that oxitropium salts 1 are suitable for effectively counteracting the side effects caused by the betamimetics 2, which occur particularly severely in the period shortly after administration. It has also been found that oxitropium salts 1 not only help to reduce the side effects which occur particularly in the period shortly after the administration of the betamimetics, but that they also significantly potentiate the desired therapeutic effect of the betamimetics 2 in a synergistic manner.

The present invention therefore relates to long-acting pharmaceutical compositions containing betamimetics 2, characterized in that they also contain oxitropium salts 1, in order to reduce the side effects caused by the administration of the betamimetics.

The present invention further relates to long-acting pharmaceutical compositions containing betamimetics 2, characterized in that they also contain oxitropium salts 1, in order to reduce the side effects caused by the betamimetics in the period shortly after the administration of the betamimetics 2.

The present invention further relates to long-acting pharmaceutical compositions containing betamimetics 2, characterized in that they also contain oxitropium salts 1 in a sufficiently high dose to reduce the side effects caused by the administration of the betamimetics.

The present invention therefore relates to long-acting pharmaceutical compositions containing betamimetics 2 characterized in that they also contain oxitropium salts 1 in order to reduce the side effects caused by the administration of the betamimetics several times a day, preferably twice a day.

The present invention further relates to long-acting pharmaceutical compositions containing betamimetics 2, characterized in that they also contain oxitropium salts 1 in order to reduce the side effects caused by the betamimetics in the period shortly after the administration of the betamimetics 2 several times a day, preferably twice a day.

The present invention further relates to long-acting pharmaceutical compositions containing betamimetics 2, characterized in that they also contain oxitropium salts 1 in a sufficiently high dose to reduce the side effects caused by the administration of the betamimetics several times a day, preferably twice a day.

In the drug combinations mentioned above, the active substances may be present either together in a single preparation or in two separate preparations. According to the invention, pharmaceutical compositions which contain the active substances 1 and 2 in a single preparation are preferred.

Salmeterol salts or formoterol salts are preferably used as the long-acting betamimetics 2 according to the invention. Any reference to the term betamimetics 2 also includes a reference to the relevant enantiomers or mixtures thereof. Any reference to the preferred compounds 2 according to the invention, the salts of salmeterol and formoterol, also includes the relevant enantiomeric salts of R-salmeterol, S-salmeterol, R,R-formoterol, S,S-formoterol, R,S-formoterol, S,R-formoterol and the mixtures thereof, while the enantiomeric salts of R-salmeterol and R,R-formoterol are of particular importance. The compounds 2 may also be present according to the invention in the form of the hydrates or solvates thereof.

Within the scope of the present invention any reference to compounds 2 is to be understood as being a reference to physiologically acceptable acid addition salts. By physiologically acceptable acid addition salts of the betamimetics 2 are meant according to the invention pharmaceutically acceptable salts which are selected from the salts of hydrochloric acid, hydrobromic acid, sulfuric acid, phosphoric acid, methanesulfonic acid, acetic acid, fumaric acid, succinic acid, lactic acid, citric acid, tartaric acid, 1-hydroxy-2-naphthalenecarboxylic acid or maleic acid. If desired, mixtures of the abovementioned acids may be used to prepare the salts 2.

According to the invention the salts of the betamimetics 2 selected from among the hydrochloride, hydrobromide, sulfate, phosphate, fumarate, methanesulfonate and xinafoate are preferred. Particularly preferred are the salts of 2 in the case of salmeterol selected from hydrochloride, sulfate and xinafoate, of which the sulfates and xinafoates are especially preferred. According to the invention salmeterol×½ H₂SO₄ and salmeterol xinafoate are of exceptional importance. Particularly preferred are the salts of 2 in the case of formoterol selected from the hydrochloride, sulfate, and fumarate, of which the hydrochloride and fumarate are particularly preferred. According to the invention formoterol fumarate is of exceptional importance.

If, within the scope of the present invention, there is a reference to betamimetics which are not in the salt form, this can be taken to mean a reference to compounds 2′. For example, the preferred betamimetics 2′ according to the invention which are not in salt form are the free base of formoterol or salmeterol, whereas the particularly preferred compounds 2 according to the invention are, for example, salmeterol xinafoate, salmeterol×½ H₂SO₄ or formoterol fumarate.

Within the scope of the present invention the betamimetics 2 are optionally also referred to as sympathomimetics or beta₂-agonists (β₂-agonists). All these names can be regarded as equivalent within the scope of the present invention.

By the oxitropium salts 1 which may be used within the scope of the present invention are meant the compounds which contain, in addition to oxitropium as counter-ion (anion), chloride, bromide, iodide, methanesulfonate, p-toluenesulfonate, or methylsulfate. Within the scope of the present invention, the methanesulfonate, chloride, bromide, and iodide are preferred of all the oxitropium salts 1, the methanesulfonate and bromide being of particular importance. Oxitropium bromide is of outstanding importance according to the invention.

The word oxitropium is intended within the scope of the present invention to refer to the free cation 1′.

In the pharmaceutical compositions according to the invention, 1 may be present in the form of its enantiomers, mixtures of enantiomers or in the form of racemates. If 1 is present in the form of an enantiomer, the preferred enantiomers are compounds 1 wherein 1′ is present in the form of the (1α,2β, 4β,5α,7β)]-9-ethyl-7-(3-hydroxy-1-oxo-2-phenylpropoxy)-9-methyl-3-oxa-9-azoniatricyclo[3.3.1.0^{2,4}]nonane

In one aspect the present invention relates to the abovementioned pharmaceutical compositions which contain, in addition to therapeutically effective quantities of 1 and 2, a pharmaceutically acceptable carrier. In one aspect the present invention relates to the abovementioned pharmaceutical compositions which do not contain any pharmaceutically acceptable carrier in addition to therapeutically effective quantities of 1 and 2.

The present invention also relates to the use of therapeutically effective quantities of oxitropium salts 1 for preparing a pharmaceutical composition containing long-acting betamimetics 2 for treating inflammatory or obstructive diseases of the respiratory tract, particularly asthma or chronic obstructive pulmonary diseases (COPD), while simultaneously reducing the stimulant effects on the heart caused by betamimetics 2, particularly tachycardia, palpitations, angina pectoris-like pain and arrhythmia.

Preferably, the present invention relates to the abovementioned use for preparing a pharmaceutical composition for treating inflammatory or obstructive diseases of the respiratory tract, particularly asthma or COPD, while simultaneously reducing the stimulant effects on the heart caused by betamimetics 2, particularly tachycardia, palpitations, angina pectoris-like pain and arrhythmia, in the period shortly after administration.

Preferably, the present invention relates to the abovementioned use for preparing a pharmaceutical composition for treating inflammatory or obstructive diseases of the respiratory tract, particularly asthma or COPD, while simultaneously reducing the stimulant effects on the heart caused by administration of the betamimetics 2 several times a day, preferably twice a day, particularly tachycardia, palpitations, angina pectoris-like pain and arrhythmia.

Preferably, the present invention relates to the abovementioned uses of oxitropium salts 1 for preparing a pharmaceutical composition for treating inflammatory or obstructive diseases of the respiratory tract, particularly asthma or COPD, while simultaneously reducing tachycardia.

Within the scope of the present invention the compounds 1 and 2 may be administered simultaneously or successively, although it is preferable according to the invention to administer compounds 1 and 2 simultaneously.

The present invention further relates to the use of therapeutically effective amounts of oxitropium salts 1 and long-acting betamimetics 2 for treating inflammatory or obstructive diseases of the respiratory tract, particularly asthma or COPD, while simultaneously reducing the stimulant effects on the heart caused by betamimetics 2, particularly tachycardia, palpitations, angina pectoris-like pain, and arrhythmia.

Preferably, the present invention relates to the abovementioned use for treating inflammatory or obstructive diseases of the respiratory tract, particularly asthma or COPD, while simultaneously reducing the stimulant effects on the heart caused by betamimetics 2, particularly tachycardia, palpitations, angina pectoris-like pain and arrhythmia, in the period shortly after administration.

Preferably, the present invention relates to the abovementioned use for treating inflammatory or obstructive diseases of the respiratory tract, particularly asthma or COPD, while simultaneously reducing the stimulant effects on the heart caused by administration of the betamimetics 2 several times a day, preferably twice a days particularly tachycardia, palpitations, angina pectoris-like pain, and arrhythmia.

Preferably, the present invention relates to the abovementioned uses of oxitropium salts 1 for preparing a pharmaceutical composition for treating inflammatory or obstructive diseases of the respiratory tract, particularly asthma or COPD, while simultaneously reducing tachycardia.

The proportions in which the two active substances 1 and 2 may be used in the active substance combinations according to the invention are variable. Active substances 1 and 2 may possibly be present in the form of their solvates or hydrates. Depending on the choice of the salts 1 and 2, the weight ratios which may be used within the scope of the present invention vary on the basis of the different molecular weights of the various salt forms. Consequently, the weight ratios given hereinafter were based on the oxitropium cation 1′ and the free bases 2′ of the preferred betamimetics according to the invention, namely salmeterol and formoterol.

The combinations of active substances according to the invention may contain 1′ and 2′ in the case of formoterol, for example, in weight ratios in the range from about 1:5 to 300:1, preferably 1:4 to 200:1, preferably 1:3 to 150:1, preferably 1:2 to 100:1, preferably 1:1 to 65:1, most preferably from 2:1 to 50:1.

For example, without limiting the scope of the invention thereto, preferred combinations of 1 and 2 according to the invention may contain oxitropium 1′ and formoterol 2′ in the following weight ratios: 1:3, 1:2, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 51:1, 52:1, 53:1, 54:1, 55:1, 56:1, 57:1, 58:1, 59:1, 60:1, 61:1, 62:1, 63:1, 64:1, 65:1, 66:1, 67:1, 68:1, 69:1, 70:1, 71:1, 72:1, 73:1, 74:1, 75:1, 76:1, 77:1, 78:1, 79:1, 80:1, 81:1, 82:1, 83:1, 84:1, and 85:1.

The pharmaceutical compositions according to the invention containing the combinations of 1 and 2 are preferably used with oxitropium 1′ and formoterol 2′ present together in doses from 5 μg to 500 0 μg, preferably 10 μg to 200 0 μg, particularly preferably 15 μg to 100 0 μg, more preferably 20 μg to 800 μg, according to the invention preferably 30 μg to 600 μg, preferably 40 μg to 500 μg, preferably 30 μg to 400 μg, preferably 40 μg to 300 μg, particularly preferably 50 μg to 250 μg per single dose.

For example, combinations of 1 and 2 according to the invention contain an amount of oxitropium 1′ and formoterol 2′ such that the total dose per single dose is about 20 μg, 25 μg, 30 μg, 35 μg, 45 μg, 50 μg, 55 μg, 60 μg, 65 μg, 70 μg, 75 μg, 80 μg, 85 μg, 90 μg, 95 μg, 100 μg, 105 μg, 110 μg, 115 μg, 120 μg, 125 μg, 130 μg, 135 μg, 140 μg, 145 μg, 150 μg, 155 μg, 160 μg, 165 μg, 170 μg, 175 μg, 180 μg, 185 μg, 190 μg, 195 μg, 200 μg, 205 μg, 210 μg, 215 μg, 220 μg, 225 μg, 230 μg, 235 μg, 240 μg, 245 μg, 250 μg, 255 μg, 260 μg, 265 μg, 270 μg, 275 μg, 280 μg, 285 μg, 290 μg, 295 μg, 300 μg, 305 μg, 310 μg, 315 μg, 320 μg, 325 μg, 330 μg, 335 μg, 340 μg, 345 μg, 350 μg, 355 μg, 360 μg, 365 μg, 370 μg, 375 μg, 380 μg, 385 μg, 390 μg, 395 μg, 400 μg, 405 μg, 410 μg, 415 μg, 420 μg, 425 μg, 430 μg, 435 μg, 440 μg, 445 μg, 450 μg, 455 μg, 460 μg, 465 μg, 470 μg, 475 μg, 480 μg, 485 μg, 490 μg, 495 μg, 500 μg, 505 μg, 510 μg, 515 μg, 520 μg or the like. It is apparent to anyone skilled in the art that the abovementioned proposed dosages per single dose should not be regarded as being limited to the numerical values explicitly stated. Fluctuations of about ±2.5 μg, particularly fluctuations in the decimal range are also included, as is apparent to anyone skilled in the art. In these dosage ranges the active substances 1′ and 2′ are present in the weight ratios described hereinbefore.

For example, and without restricting the scope of the invention thereto, the combinations of 1 and 2 according to the invention may contain an amount of oxitropium 1′ and formoterol 2′ such that for example 16.1 μg of 1′ and 4.9 μg of 2′, 16.1 μg of 1′ and 9.8 μg of 2′, 16.1 μg of 1′ and 14.7 μg of 2′, 16.1 μg of 1′ and 19.6 μg of 2′, 16.1 μg of 1′ and 24.4 μg of 2′, 32.2 μg of 1′ and 4.9 μg of 2′, 32.2 μg of 1′ and 9.8 μg of 2′, 32.2 μg of 1′ and 14.7 μg of 2′, 32.2 μg of 1′ and 19.6 μg of 2′, 32.2 μg of 1′ and 24.4 μg of 2′, 48.3 μg of 1′ and 4.9 μg of 2′, 48.3 μg of 1′ and 9.8 μg of 2′, 48.3 μg of 1′ and 14.7 μg of 2′, 48.3 μg of 1′ and 19.6 μg of 2′, 48.3 μg of 1′ and 24.4 μg of 2′, 80.5 μg of 1′ and 4.9 μg of 2′, 80.5 μg of 1′ and 9.8 μg of 2′, 80.5 μg of 1′ and 14.7 μg of 2′, 80.5 μg of 1′ and 19.6 μg of 2′, 80.5 μg of 1′ and 24.4 μg of 2′, 161 μg of 1′ and 4.9 μg of 2′, 161 μg of 1′ and 9.8 μg of 2′, 161 μg of 1′ and 14.7 μg of 2′, 161 μg of 1′ and 19.6 μg of 2′, 161 μg of 1′ and 24.4 μg of 2′, 201.5 μg of 1′ and 4.9 μg of 2′, 201.5 μg of 1′ and 9.8 μg of 2′, 201.5 g of 1′ and 14.7 μg of 2′, 201.5 μg of 1′ and 19.6 μg of 2′, 201.5 μg of 1′ and 24.4 μg of 2′, 403 μg of 1′ and 4.9 μg of 2′, 403 μg of 1′ and 9.8 μg of 2′, 403 μg of 1′ and 14.7 μg of 2′, 403 μg of 1′ and 19.6 μg of 2′, 403 μg of 1′ and 24.4 μg of 2′ are present per single dose.

If as the preferred combination of 1 and 2 according to the invention the active substance combination is used wherein 1 denotes oxitropium bromide and 2 denotes formoterol fumarate, the quantities of active substance 1′ and 2′ administered per single dose as mentioned above correspond to the following amounts of 1 and 2 administered per single dose: 20 μg of 1 and 5.7 μg of 2, 20 μg of 1 and 11.5 μg of 2, 20 μg of 1 and 17.2 μg of 2, 20 μg of 1 and 22.9 μg of 2, 20 μg of 1 and 28.5 μg of 2, 40 μg of 1 and 5.7 μg of 2, 40 μg of 1 and 11.5 μg of 2, 40 μg of 1 and 17.2 μg of 2, 40 μg of 1 and 22.9 μg of 2, 40 μg of 1 and 28.5 μg of 2, 60 μg of 1 and 5.7 μg of 2, 60 μg of 1 and 11.5 μg of 2, 60 μg of 1 and 17.2 μg of 2, 60 μg of 1 and 22.9 μg of 2, 60 μg of 1 and 28.5 μg of 2, 100 μg of 1 and 5.7 μg of 2, 100 μg of 1 and 11.5 μg of 2, 100 μg of 1 and 17.2 μg of 2, 100 μg of 1 and 22.9 μg of 2, 100 μg of 1 and 28.5 μg of 2, 200 μg of 1 and 5.7 μg of 2, 200 μg of 1 and 11.5 μg of 2, 200 μg of 1 and 17.2 μg of 2, 200 μg of 1 and 22.9 μg of 2, 200 μg of 1 and 28.5 μg of 2, 250 μg of 1 and 5.7 μg of 2, 250 μg of 1 and 11.5 μg of 2, 250 μg of 1 and 17.2 μg of 2, 250 μg of 1 and 22.9 μg of 2, 250 μg of 1 and 28.5 μg of 2, 500 μg of 1 and 5.7 μg of 2, 500 μg of 1 and 11.5 μg of 2, 500 μg of 1 and 17.2 μg of 2, 500 μg of 1 and 22.9 μg of 2, 500 μg of 1 and 28.5 μg of 2.

If the preferred combination of 1 and 2 used according to the invention is the active substance combination wherein 2 denotes formoterol fumarate dihydrate and if oxitropium bromide monohydrate is used as 1, for example, the quantities of active substance 1′ and 2′ administered per single dose as mentioned above by way of example correspond to the following amounts of 1 and 2 administered per single dose. 20.8 μg of 1 and 6 μg of 2, 20.8 μg of 1 and 12 μg of 2, 20.8 μg of 1 and 18 μg of 2, 20.8 μg of 1 and 24 μg of 2, 20.8 μg of 1 and 30 μg of 2, 41.7 μg of 1 and 6 μg of 2, 41.7 μg of 1 and 12 μg of 2, 417 μg of 1 and 18 μg of 2, 41.7 μg of 1 and 24 μg of 2, 41.7 μg of 1 and 30 μg of 2, 62.5 μg of 1 and 6 μg of 2, 62.5 μg of 1 and 12 μg of 2, 62.5 μg of 1 and 18 μg of 2, 62.5 μg of 1 and 24 μg of 2, 62.5 μg of 1 and 30 μg of 2, 104.2 μg of 1 and 6 μg of 2, 104.2 μg of 1 and 12 μg of 2, 104.2 μg of 1 and 18 μg of 2, 104.2 μg of 1 and 24 μg of 2, 104.2 μg of 1 and 30 μg of 2, 208.3 μg of 1 and 6 μg of 2, 208.3 μg of 1 and 12 μg of 2, 208.3 μg of 1 and 18 μg of 2, 208.3 μg of 1 and 24 μg of 2, 208.3 μg of 1 and 30 μg of 2, 260.7 μg of 1 and 6 μg of 2, 260.7 μg of 1 and 12 μg of 2, 260.7 μg of 1 and 18 μg of 2, 260.7 μg of 1 and 24 μg of 2, 260.7 μg of 1 and 30 g of 2, 521.5 g of 1 and 6 μg of 2, 521.5 g of 1 and 12 μg of 2, 521.5 μg of 1 and 18 μg of 2, 521.5 μg of 1 and 24 g of 2, 521.5 μg of 1 and 30 μg of 2.

The active substance combinations according to the invention may contain 1′ and 2′ in the case of salmeterol, for example, in weight ratios in the range from about 1:30 to 400:1, preferably 1:25 to 200:1, preferably 1:20 to 100:1, preferably 1:15 to 50:1, preferably 1:13 to 20:1, preferably 1:1 to 15:1, for example.

For example, and without restricting the scope of the invention thereto, preferred combinations of 1 and 2 according to the invention may contain oxitropium 1′ and salmeterol 2′ in the following weight ratios: 1:15, 1:14, 1:13, 1:12, 1:11, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1.

The pharmaceutical compositions according to the invention containing the combinations of 1 and 2 are preferably administered so that oxitropium 1′ and salmeterol 2′ are given together in doses from 5 μg to 500 0 μg, preferably from 10 μg to 200 0 μg, particularly preferably 15 μg to 100 0 μg, more preferably 20 μg to 800 μg, preferably according to the invention 30 μg to 700 μg, preferably from 40 μg to 600 μg, preferably 50 μg to 550 μg, preferably 40 μg to 500 μg, particularly preferably 50 μg to 400 μg per single dose.

For example, combinations of 1 and 2 according to the invention contain a quantity of oxitropium 1′ and salmeterol 2′ such that the total dosage per single dose is about 35 μg, 45 μg, 50 μg, 55 μg, 60 μg, 65 μg, 70 μg, 75 μg, 80 μg, 85 μg, 90 μg, 95 μg, 100 μg, 105 μg, 110 μg, 115 μg, 120 μg, 125 μg, 130 μg, 135 g, 140 μg, 145 μg, 150 μg, 155 μg, 160 μg, 165 μg, 170 μg, 175 μg, 180 μg, 185 μg, 190 μg, 195 μg, 200 μg, 205 μg, 210 μg, 215 μg, 220 μg, 225 μg, 230 μg, 235 μg, 240 μg, 245 μg, 250 μg, 255 μg, 260 μg, 265 μg, 270 μg, 275 μg, 280 μg, 285 μg, 290 μg, 295 μg, 300 μg, 305 μg, 310 μg, 315 μg, 320 μg, 325 μg, 330 μg, 335 μg, 340 μg, 345 μg, 350 μg, 355 μg, 360 μg, 365 μg, 370 μg, 375 μg, 380 μg, 385 μg, 390 μg, 395 μg, 400 μg, 405 μg, 410 μg, 415 μg, 420 μg, 425 μg, 430 μg, 435 μg, 440 μg, 445 μg, 450 μg, 455 μg, 460 μg, 465 μg, 470 μg, 475 μg, 480 μg, 485 μg, 490 μg, 495 μg, 500 μg, 505 μg, 510 μg, 515 μg, 520 μg, 525 μg, 530 μg, 535 μg, 540 μg, 545 μg, 550 μg, 555 μg, 560 μg, 565 μg, 570 μg, 575 μg, 580 μg, 585 μg, 590 μg, 595 μg, 600 μg, 605 μg, 610 μg, or the like. It is clear to the skilled man that the above suggestions of dosages per single dose should not be regarded as being restricted to the numerical values specifically stated. Fluctuations of about ±2.5 μg, particularly fluctuations in the decimal range are also included, as is apparent to anyone skilled in the art. In these dosage ranges the active substances 1′ and 2′ are present in the weight ratios described hereinbefore.

For example, and without restricting the scope of the invention thereto, the combinations of 1 and 2 according to the invention may contain an amount of oxitropium 1′ and formoterol 2′ such that for example 16.1 μg of 1 and 25 μg of 2′, 16.1 μg of 1′ and 50 μg of 2′, 16.1 μg of 1′ and 75 μg of 2′, 16.1 g of 1′ and 100 μg of 2′, 16.1 μg of 1′ and 200 μg of 2′, 32.2 g of 1′ and 25 μg of 2′, 32.2 μg of 1′ and 50 μg of 2′, 32.2 μg of 1′ and 75 μg of 2′, 32.2 μg of 1′ and 100 μg of 2′, 32.2 μg of 1′ and 200 μg of 2′, 48.3 μg of 1′ and 25 μg of 2′, 48.3 μg of 1′ and 50 μg of 2′, 48.3 μg of 1′ and 75 μg of 2′, 48.3 μg of 1′ and 100 μg of 2′, 48.3 μg of 1′ and 200 μg of 2′, 80.5 μg of 1′ and 25 μg of 2′, 80.5 μg of 1′ and 50 μg of 2′, 80.5 μg of 1′ and 75 g of 2′, 80.5 μg of 1′ and 100 μg of 2′, 80.5 μg of 1′ and 200 μg of 2′, 161 μg of 1′ and 25 μg of 2′, 161 μg of 1′ and 50 μg of 2′, 161 μg of 1′ and 75 μg of 2′, 161 μg of 1′ and 100 μg of 2′, 161 μg of 1′ and 200 μg of 2′, 201.5 μg of 1′ and 25 μg of 2′, 201.5 μg of 1′ and 50 μg of 2′, 201.5 μg of 1′ and 75 μg of 2′, 201.5 μg of 1′ and 100 μg of 2′, 201.5 μg of 1′ and 200 μg of 2′, 403 μg of 1′ and 25 μg of 2′, 403 μg of 1′ and 50 μg of 2′, 403 μg of 1′ and 75 μg of 2′, 403 μg of 1′ and 100 μg of 2′, 403 g of 1′ and 200 μg of 2′ are present per single dose.

If as the preferred combination of 1 and 2 according to the invention the active substance combination is used wherein 1 denotes oxitropium bromide and 2 denotes salmeterol xinafoate, the quantities of active substance 1′ and 2′ administered per single dose as mentioned above by way of example correspond to the following amounts of 1 and 2 administered per single dose: 20 μg of 1 and 36.3 μg of 2, 20 μg of 1 and 72.6 μg of 2, 20 μg of 1 and 108.9 μg of 2, 20 μg of 1 and 145.2 μg of 2, 20 μg of 1 and 290.4 μg of 2, 40 μg of 1 and 36.3 μg of 2, 40 μg of 1 and 72.6 μg of 2, 40 μg of 1 and 108.9 μg of 2, 40 μg of 1 and 145.2 μg of 2, 40 μg of 1 and 290.4 μg of 2, 60 μg of 1 and 36.3 μg of 2, 60 μg of 1 and 72.6 μg of 2, 60 μg of 1 and 108.9 μg of 2, 60 μg of 1 and 145.2 μg of 2, 60 μg of 1 and 290.4 μg of 2, 100 μg of 1 and 36.3 μg of 2, 100 μg of 1 and 72.6 μg of 2, 100 μg of 1 and 108.9 μg of 2, 100 μg of 1 and 145.2 μg of 2, 100 μg of 1 and 290.4 μg of 2, 200 μg of 1 and 36.3 μg of 2, 200 μg of 1 and 72.6 μg of 2, 200 μg of 1 and 108.9 μg of 2, 200 μg of 1 and 145.2 μg of 2, 200 μg of 1 and 290.4 μg of 2, 250 μg of 1 and 36.3 μg of 2, 250 μg of 1 and 72.6 μg of 2, 250 μg of 1 and 108.9 μg of 2, 250 μg of 1 and 145.2 μg of 2, 250 μg of 1 and 290.4 μg of 2, 500 μg of 1 and 36.3 μg of 2, 500 μg of 1 and 72.6 μg of 2, 500 μg of 1 and 108.9 μg of 2, 500 μg of 1 and 145.2 μg of 2, 500 μg of 1 and 290.4 μg of 2.

If as the preferred combination of 1 and 2 according to the invention the active substance combination is used wherein 1 denotes oxitropium bromide monohydrate and 2 denotes salmeterol xinafoate, the quantities of active substance 1′ and 2′ administered per single dose as mentioned above by way of example correspond to the following amounts of 1 and 2 administered per single dose: 20.8 μg of 1 and 36.3 μg of 2, 20.8 μg of 1 and 72.6 μg of 2, 20.8 μg of 1 and 108.9 μg of 2, 20.8 μg of 1 and 145.2 μg of 2, 20.8 μg of 1 and 290.4 μg of 2, 41.7 μg of 1 and 36.3 μg of 2, 41.7 μg of 1 and 72.6 μg of 2, 41.7 μg of 1 and 108.9 μg of 2, 41.7 μg of 1 and 145.2 μg of 2, 41.7 μg of 1 and 290.4 μg of 2, 62.5 μg of 1 and 36.3 μg of 2, 62.5 μg of 1 and 72.6 μg of 2, 62.5 μg of 1 and 108.9 μg of 2, 62.5 μg of 1 and 145.2 μg of 2, 62.5 μg of 1 and 290.4 μg of 2, 104.2 μg of 1 and 36.3 μg of 2, 104.2 μg of 1 and 72.6 μg of 2, 104.2 μg of 1 and 108.9 μg of 2, 104.2 μg of 1 and 145.2 μg of 2, 104.2 μg of 1 and 290.4 μg of 2, 208.3 μg of 1 and 36.3 μg of 2, 208.3 μg of 1 and 72.6 μg of 2, 208.3 μg of 1 and 108.9 μg of 2, 208.3 μg of 1 and 145.2 μg of 2, 208.3 μg of 1 and 290.4 μg of 2, 260.7 μg of 1 and 36.3 μg of 2, 260.7 μg of 1 and 72.6 μg of 2, 260.7 μg of 1 and 108.9 μg of 2, 260.7 μg of 1 and 145.2 μg of 2, 260.7 μg of 1 and 290.4 μg of 2, 521.5 μg of 1 and 36.3 μg of 2, 521.5 μg of 1 and 72.6 μg of 2, 521.5 μg of 1 and 108.9 μg of 2, 521.5 μg of 1 and 145.2 μg of 2, 521.5 μg of 1 and 290.4 μg of 2.

The amounts of active substance administered per single dose in the drug combinations according to the invention may be calculated analogously if salmeterol-1/2-sulfate is used instead of salmeterol xinafoate as compound 2 according to the invention.

The active substance combinations of 1 and 2 according to the invention are preferably administered by inhalation. For this purpose, ingredients 1 and 2 have to be made available in forms suitable for inhalation. Inhalable preparations include inhalable powders, propellant-containing metering aerosols or propellant-free inhalable solutions. Inhalable powders according to the invention containing the combination of active substances 1 and 2 may consist of the active substances on their own or of a mixture of the active substances with physiologically acceptable excipients. Within the scope of the present invention, the term propellant-free inhalable solutions also includes concentrates or sterile inhalable solutions ready for use. The preparations according to the invention may contain the combination of active substances 1 and 2 either together in one formulation or in two separate formulations. These formulations which may be used within the scope of the present invention are described in more detail in the next part of the specification.

A. Inhalable Powder Containing the Combinations of Active Substances 1 and 2 According to the Invention

The inhalable powders according to the invention may contain 1 and 2 either on their own or in admixture with suitable physiologically acceptable excipients. If the active substances 1 and 2 are present in admixture with physiologically acceptable excipients, the following physiologically acceptable excipients may be used to prepare these inhalable powders according to the invention: monosaccharides (e.g., glucose or arabinose), disaccharides (e.g., lactose, saccharose, maltose), oligo- and polysaccharides (e.g., dextrane), polyalcohols (e.g., sorbitol, mannitol, xylitol), salts (e.g., sodium chloride, calcium carbonate) or mixtures of these excipients with one another. Preferably, mono- or disaccharides are used, while the use of lactose or glucose is preferred, particularly, but not exclusively, in the form of their hydrates.

Within the scope of the inhalable powders according to the invention the excipients have a maximum average particle size of up to 250 μm, preferably between 10 μm and 150 μm, most preferably between 15 μm and 80 μm, It may sometimes seem appropriate to add finer excipient fractions with an average particle size of 1 μm to 9 μm to the excipient mentioned above. These finer excipients are also selected from the group of possible excipients listed hereinbefore. Finally, in order to prepare the inhalable powders according to the invention, micronised active substance 1 and 2, preferably with an average particle size of 0.5 μm to 10 μm, more preferably from 1 μm to 6 μm, is added to the excipient mixture. Processes for producing the inhalable powders according to the invention by grinding and micronizing and by finally mixing the ingredients together are known from the prior art. The inhalable powders according to the invention may be prepared and administered either in the form of a single powder mixture which contains both 1 and 2 or in the form of separate inhalable powders which contain only 1 or 2.

The inhalable powders according to the invention may be administered using inhalers known from the prior art. Inhalable powders according to the invention which contain a physiologically acceptable excipient in addition to 1 and 2 may be administered, for example, by means of inhalers which deliver a single dose from a supply using a measuring chamber as described in U.S. Pat. No. 4,570,630 A, or by other means as described in DE 36 25685 A. The inhalable powders according to the invention which contain 1 and 2 optionally in conjunction with a physiologically acceptable excipient may be administered, for example, using the inhaler known by the name Turbuhalerg® or using inhalers as disclosed for example in EP 237507 A. Preferably, the inhalable powders according to the invention which contain physiologically acceptable excipient in addition to 1 and 2 are packed into capsules (to produce so-called inhalettes) which are used in inhalers as described, for example, in WO 94/28958.

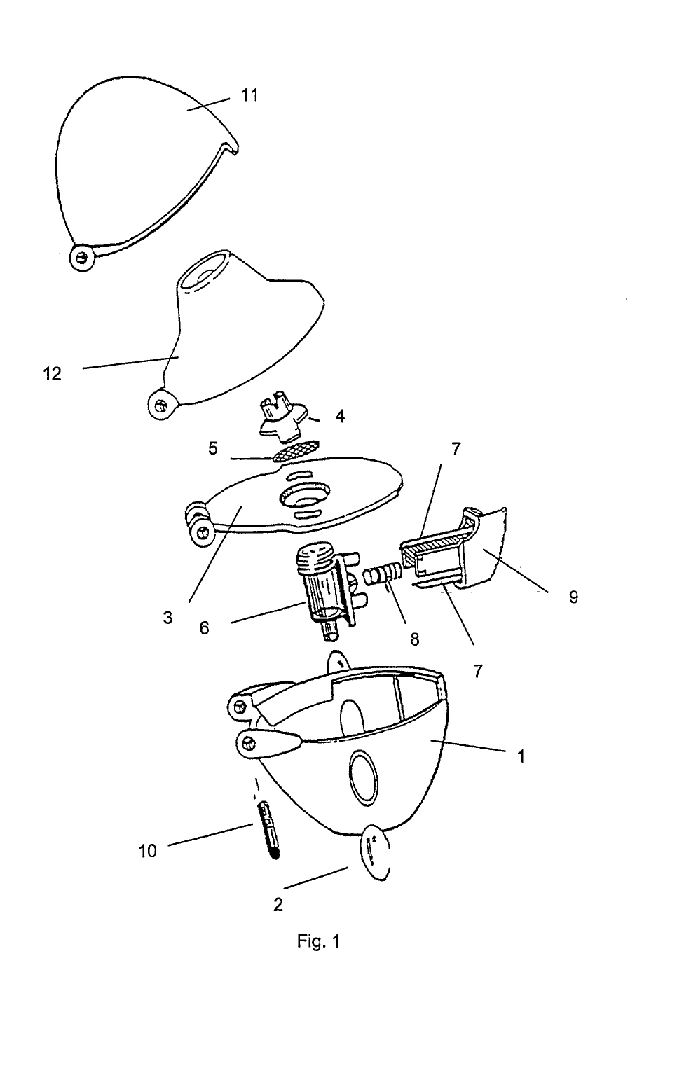

A particularly preferred inhaler for using the pharmaceutical combination according to the invention in inhalettes is shown in FIG. 1.

This inhaler (HANDIHALER®) for inhaling powdered pharmaceutical compositions from capsules is characterized by a housing (1) containing two windows (2), a deck (3) in which there are air inlet portions and which is provided with a screen (5) secured via a screen housing (4), an inhalation chamber (6) connected to the deck (3) on which there is a push button (9) provided with two sharpened pins (7) and movable counter to a spring (8), and a mouthpiece (12) which is connected to the housing (1), the deck (3) and a cover (11) via a spindle (10) to enable it to be flipped open or shut.

If the inhalable powders according to the invention are packed into capsules (inhalers) for the preferred use described above, the quantities packed into each capsule should be 1 mg to 30 mg per capsule. These capsules contain, according to the invention, either together or separately, the doses of 1 or 1′ and 2 or 2′ mentioned hereinbefore for each single dose.

B. Propellant Gas-driven Inhalation Aerosols Containing the Combinations of Active Substances 1 and 2

Inhalation aerosols containing propellant gas according to the invention may contain substances 1 and 2 dissolved in the propellant gas or in dispersed form. 1 and 2 may be present in separate formulations or in a single preparation, in which 1 and 2 are either both dissolved, both dispersed or only one component is dissolved and the other is dispersed. The propellant gases which may be used to prepare the inhalation aerosols according to the invention are known from the prior art. Suitable propellant gases are selected from among hydrocarbons such as n-propane, n-butane, or isobutane and halohydrocarbons such as preferably chlorinated and fluorinated derivatives of methane, ethane, propane, butane, cyclopropane or cyclobutane. The propellant gases mentioned above may be used on their own or in mixtures thereof. Particularly preferred propellant gases are halogenated alkane derivatives selected from TG11, TG12, TG134a (1,1,1,2-tetrafluoroethane), TG227 (1,1,1,2,3,3,3-heptafluoropropane) and mixtures thereof, the propellant gases TG134a, TG227 and mixtures thereof being preferred.

The propellant-driven inhalation aerosols according to the invention may also contain other ingredients such as co-solvents, stabilizers, surfactants, antioxidants, lubricants and pH adjusters. All these ingredients are known in the art.

The inhalation aerosols containing propellant gas according to the invention may contain up to 5 wt. % of active substance 1 and/or 2. Aerosols according to the invention contain, for example, 0.002 wt. % to 5 wt. %, 0.01 wt. % to 3 wt. %, 0.015 wt. % to 2 wt. %, 0.1 wt. % to 2 wt. %, 0.5 wt. % to 2 wt. % or 0.5 wt. % to 1 wt. % of active substance 1 and/or 2.

If the active substances 1 and/or 2 are present in dispersed form, the particles of active substance preferably have an average particle size of up to 10 μm, preferably from 0.1 μm to 5 μm, more preferably from 1 μm to 5 μm.

The propellant-driven inhalation aerosols according to the invention mentioned above may be administered using inhalers known in the art (MDIs=metered dose inhalers). Accordingly, in another aspect, the present invention relates to pharmaceutical compositions in the form of propellant-driven aerosols as hereinbefore described combined with one or more inhalers suitable for administering these aerosols. In addition, the present invention relates to inhalers which are characterized in that they contain the propellant gas-containing aerosols described above according to the invention. The present invention also relates to cartridges which are fitted with a suitable valve and can be used in a suitable inhaler and which contain one of the above-mentioned propellant gas-containing inhalation aerosols according to the invention. Suitable cartridges and methods of filling these cartridges with the inhalable aerosols containing propellant gas according to the invention are known from the prior art.

C. Propellant-free Inhalable Solutions or Suspensions Containing the Combinations of Active Substances 1 and 2 According to the Invention

It is particularly preferred to use the active substance combination according to the invention in the form of propellant-free inhalable solutions and suspensions if salmeterol-1/2-sulfate is used as the betamimetic 2 in the drug combinations according to the invention. The solvent used may be an aqueous or alcoholic, preferably an ethanolic solution. The solvent may be water on its own or a mixture of water and ethanol. The relative proportion of ethanol compared with water is not limited but the maximum is up to 70 percent by volume, more particularly up to 60 percent by volume and most preferably up to 30 percent by volume. The remainder of the volume is made up of water. The solutions or suspensions containing 1 and 2, separately or together, are adjusted to a pH of 2 to 7, preferably 2 to 5, using suitable acids. The pH may be adjusted using acids selected from inorganic or organic acids. Examples of suitable inorganic acids include hydrochloric acid, hydrobromic acid, nitric acid, sulfuric acid and/or phosphoric acid. Examples of particularly suitable organic acids include ascorbic acid, citric acid, malic acid, tartaric acid, maleic acid, succinic acid, fimaric acid, acetic acid, formic acid and/or propionic acid etc. Preferred inorganic acids are hydrochloric and sulfuric acids. It is also possible to use the acids which have already formed an acid addition salt with one of the active substances. Of the organic acids, ascorbic acid, fumaric acid and citric acid are preferred. If desired, mixtures of the above acids may be used, particularly in the case of acids which have other properties in addition to their acidifying qualities, e.g., as flavorings, antioxidants or complexing agents, such as citric acid or ascorbic acid, for example. According to the invention, it is particularly preferred to use hydrochloric acid to adjust the pH.

According to the invention, the addition of edetic acid (EDTA) or one of the known salts thereof, sodium edetate, as stabilizer or complexing agent is unnecessary in the present formulation. Other embodiments may contain this compound or these compounds. In a preferred embodiment the content based on sodium edetate is less than 100 mg/100 mL, preferably less than 50 mg/mL, more preferably less than 20 mg/mL. Generally, inhalable solutions in which the content of sodium edetate is from 0 to 10 mg/100 mL are preferred.

Co-solvents and/or other excipients may be added to the propellant-free inhalable solutions according to the invention. Preferred co-solvents are those which contain hydroxyl groups or other polar groups, e.g., alcohols—particularly isopropyl alcohol, glycols—particularly propyleneglycol, polyethyleneglycol, polypropylene glycol, glycol ether, glycerol, polyoxyethylene alcohols and polyoxyethylene fatty acid esters. The terms excipients and additives in this context denote any pharmacologically acceptable substance which is not an active substance but which can be formulated with the active substance or substances in the pharmacologically suitable solvent in order to improve the qualitative properties of the active substance formulation. Preferably, these substances have no pharmacological effect or, in connection with the desired therapy, no appreciable or at least no undesirable pharmacological effect. The excipients and additives include, for example, surfactants such as soya lecithin, oleic acid, sorbitan esters, such as polysorbates, polyvinylpyrrolidone, other stabilizers, complexing agents, antioxidants and/or preservatives which guarantee or prolong the shelf life of the finished pharmaceutical formulation, flavorings, vitamins and/or other additives known in the art. The additives also include pharmacologically acceptable salts such as sodium chloride as isotonic agents.

The preferred excipients include antioxidants such as ascorbic acid, for example, provided that it has not already been used to adjust the pH, vitamin A, vitamin E, tocopherols and similar vitamins and provitamins occurring in the human body.

Preservatives may be used to protect the formulation from contamination with pathogens. Suitable preservatives are those which are known in the art, particularly cetyl pyridinium chloride, benzalkonium chloride or benzoic acid or benzoates such as sodium benzoate in the concentration known from the prior art. The preservatives mentioned above are preferably present in concentrations of up to 50 mg/100 mL, more preferably between 5 and 20 mg/100 mL.

Preferred formulations contain, in addition to the solvent water and the combination of active substances 1 and 2, only benzalkonium chloride and sodium edetate. In another preferred embodiment, no sodium edetate is present.

The propellant-free inhalable solutions according to the invention are administered in particular using inhalers of the kind which are capable of nebulizing a small amount of a liquid formulation in the therapeutic dose within a few seconds to produce an aerosol suitable for therapeutic inhalation. Within the scope of the present invention, preferred inhalers are those in which a quantity of less than 100 μL, preferably less than 50 μL, more preferably between 10 μL and 30 μL of active substance solution can be nebulized in preferably one spray action to form an aerosol with an average particle size of less than 20 μm, preferably less than 10 μm, in such a way that the inhalable part of the aerosol corresponds to the therapeutically effective quantity.

An apparatus of this kind for propellant-free delivery of a metered quantity of a liquid pharmaceutical composition for inhalation is described for example in International Patent Application WO 91/14468 and also in WO 97/12687 (cf in particular FIGS. 6a and 6b). The nebulizers (devices) described therein are known by the name RESPIMAT®.

This RESPIMAT® nebulizer can advantageously be used to produce the inhalable aerosols according to the invention containing the combination of active substances 1 and 2. Because of its cylindrical shape and handy size of less than 9 to 15 cm long and 2 to 4 cm wide, this device can be carried at all times by the patient. The nebulizer sprays a defined volume of pharmaceutical formulation using high pressures through small nozzles so as to produce inhalable aerosols.

The preferred atomizer essentially consists of an upper housing part, a pump housing, a nozzle, a locking mechanism, a spring housing, a spring and a storage container, characterized by

a pump housing which is secured in the upper housing part and which comprises at one end a nozzle body with the nozzle or nozzle arrangement,

a hollow plunger with valve body,

a power takeoff flange in which the hollow plunger is secured and which is located in the upper housing part,

a locking mechanism situated in the upper housing part,

a spring housing with the spring contained therein, which is rotatably mounted on the upper housing part by means of a rotary bearing,

a lower housing part which is fitted onto the spring housing in the axial direction.

The hollow plunger with valve body corresponds to a device disclosed in WO 97/12687. It projects partially into the cylinder of the pump housing and is axially movable within the cylinder. Reference is made in particular to FIGS. 1 to 4, especially FIG. 3, and the relevant parts of the description. The hollow plunger with valve body exerts a pressure of 5 MPa to 60 MPa (about 50 bar to 600 bar), preferably 10 MPa to 60 MPa (about 100 bar to 600 bar) on the fluid, the measured amount of active substance solution, at its high pressure end at the moment when the spring is actuated. Volumes of 10 microliters to 50 microliters are preferred, while volumes of 10 microliters to 20 microliters are particularly preferred and a volume of 15 microliters per spray is most particularly preferred.

The valve body is preferably mounted at the end of the hollow plunger facing the valve body.

The nozzle in the nozzle body is preferably microstructured, i.e., produced by microtechnology. Microstructured valve bodies are disclosed for example in WO 94/07607; reference is hereby made to the contents of this specification, particularly FIG. 1 therein and the associated description.

The valve body consists for example of two sheets of glass and/or silicon firmly joined together, at least one of which has one or more microstructured channels which connect the nozzle inlet end to the nozzle outlet end. At the nozzle outlet end there is at least one round or non-round opening 2 microns to 10 microns deep and 5 microns to 15 microns wide, the depth preferably being 4.5 microns to 6.5 microns while the length is preferably 7 microns to 9 microns.

In the case of a plurality of nozzle openings, preferably two, the directions of spraying of the nozzles in the nozzle body may extend parallel to one another or may be inclined relative to one another in the direction of the nozzle opening. In a nozzle body with at least two nozzle openings at the outlet end the directions of spraying may be at an angle of 20° to 160° to one another, preferably 60° to 150°, most preferably 80° to 100°. The nozzle openings are preferably arranged at a spacing of 10 to 200 microns, more preferably at a spacing of 10 microns to 100 microns, most preferably 30 microns to 70 microns. Spacings of 50 microns are most preferred. The directions of spraying will therefore meet in the vicinity of the nozzle openings.

The liquid pharmaceutical preparation strikes the nozzle body with an entry pressure of up to 600 bar, preferably 200 bar to 300 bar, and is atomized into an inhalable aerosol through the nozzle openings. The preferred particle or droplet sizes of the aerosol are up to 20 microns, preferably 3 microns to 10 microns.

The locking mechanism contains a spring, preferably a cylindrical helical compression spring, as a store for the mechanical energy. The spring acts on the power takeoff flange as an actuating member the movement of which is determined by the position of a locking member. The travel of the power takeoff flange is precisely limited by an upper and lower stop. The spring is preferably biased, via a power step-up gear, e.g., a helical thrust gear, by an external torque which is produced when the upper housing part is rotated counter to the spring housing in the lower housing part. In this case, the upper housing part and the power takeoff flange have a single or multiple V-shaped gear.

The locking member with engaging locking surfaces is arranged in a ring around the power takeoff flange. It consists, for example, of a ring of plastic or metal which is inherently radially elastically deformable. The ring is arranged in a plane at right angles to the atomizer axis. After the biasing of the spring, the locking surfaces of the locking member move into the path of the power takeoff flange and prevent the spring from relaxing. The locking member is actuated by means of a button. The actuating button is connected or coupled to the locking member. In order to actuate the locking mechanism, the actuating button is moved parallel to the annular plane, preferably into the atomizer; this causes the deformable ring to deform in the annual plane. Details of the construction of the locking mechanism are given in WO 97/20590.

The lower housing part is pushed axially over the spring housing and covers the mounting, the drive of the spindle and the storage container for the fluid.

When the atomizer is actuated the upper housing part is rotated relative to the lower housing part, the lower housing part taking the spring housing with it. The spring is thereby compressed and biased by means of the helical thrust gear and the locking mechanism engages automatically. The angle of rotation is preferably a whole-number fraction of 360°, e.g., 180°. At the same time as the spring is biased, the power takeoff part in the upper housing part is moved along by a given distance, the hollow plunger is withdrawn inside the cylinder in the pump housing, as a result of which some of the fluid is sucked out of the storage container and into the high pressure chamber in front of the nozzle.

If desired, a number of exchangeable storage containers which contain the fluid to be atomized may be pushed into the atomizer one after another and used in succession. The storage container contains the aqueous aerosol preparation according to the invention.

The atomizing process is initiated by pressing gently on the actuating button. As a result, the locking mechanism opens up the path for the power takeoff member. The biased spring pushes the plunger into the cylinder of the pump housing. The fluid leaves the nozzle of the atomizer in atomized form.

Further details of construction are disclosed in PCT Applications WO 97/12683 and WO 97/20590, to which reference is hereby made, and each of which is incorporated herein by reference in their entireties.

The components of the atomizer (nebulizer) are made of a material which is suitable for its purpose. The housing of the atomizer and, if its operation permits, other parts as well are preferably made of plastics, e.g., by injection moulding. For medicinal purposes, physiologically safe materials are used.

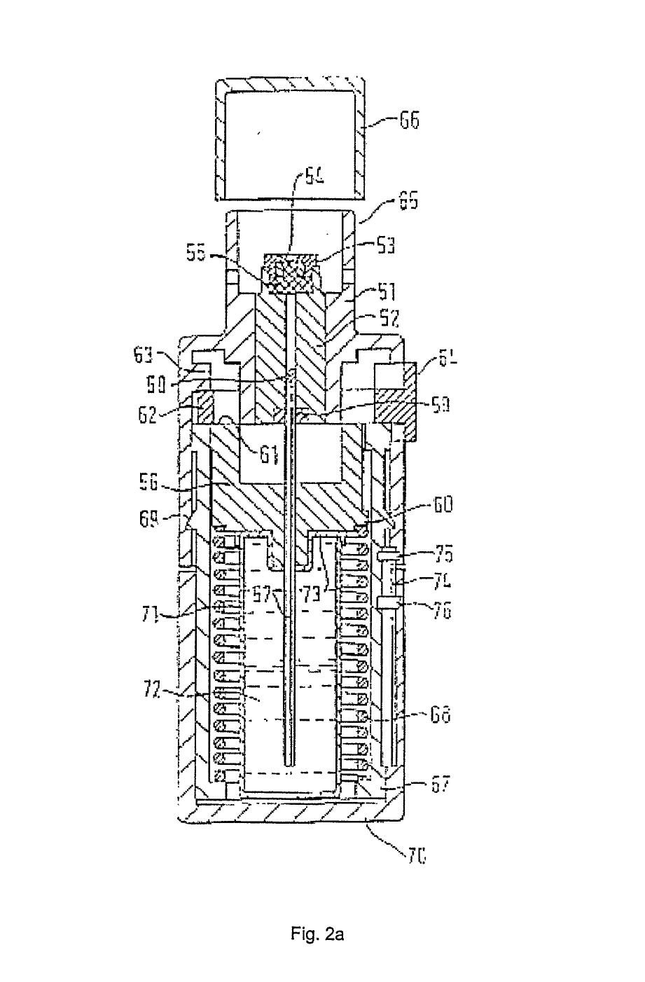

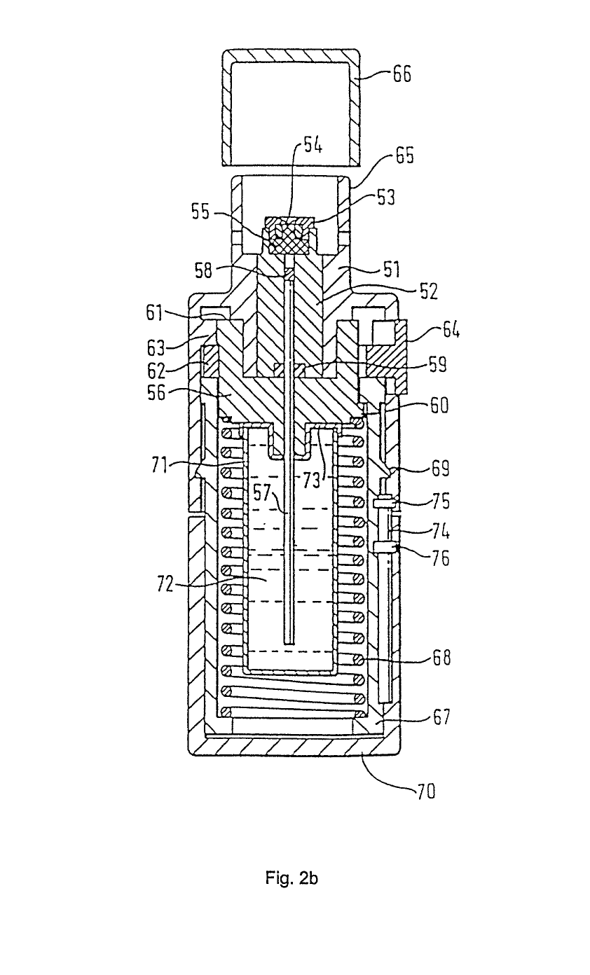

FIGS. 2a and 2b attached to this patent application, which are identical to FIGS. 6a and 6b of WO 97/12687, show the RESPIMAT® nebulizer which can advantageously be used for inhaling the aqueous aerosol preparations according to the invention.

FIG. 2a shows a longitudinal section through the atomizer with the spring biased while FIG. 2b shows a longitudinal section through the atomizer with the spring relaxed.

The upper housing part (51) contains the pump housing (52) on the end of which is mounted the holder (53) for the atomizer nozzle. In the holder is the nozzle body (54) and a filter (55). The hollow plunger (57) fixed in the power takeoff flange (56) of the locking mechanism projects partially into the cylinder of the pump housing. At its end the hollow plunger carries the valve body (58). The hollow plunger is sealed off by means of the seal (59). Inside the upper housing part is the stop (60) on which the power takeoff flange abuts when the spring is relaxed. On the power takeoff flange is the stop (61) on which the power takeoff flange abuts when the spring is biased. After the biasing of the spring the locking member (62) moves between the stop (61) and a support (63) in the upper housing part. The actuating button (64) is connected to the locking member. The upper housing part ends in the mouthpiece (65) and is sealed off by means of the protective cover (66) which can be placed thereon.

The spring housing (67) with compression spring (68) is rotatably mounted on the upper housing part by means of the snap-in lugs (69) and rotary bearing. The lower housing part (70) is pushed over the spring housing. Inside the spring housing is the exchangeable storage container (71) for the fluid (72) which is to be atomized. The storage container is sealed off by the stopper (73) through which the hollow plunger projects into the storage container and is immersed at its end in the fluid (supply of active substance solution).

The spindle (74) for the mechanical counter is mounted in the covering of the spring housing. At the end of the spindle facing the upper housing part is the drive pinion (75). The slider (76) sits on the spindle.

The nebulizer described above is suitable for nebulizing the aerosol preparations according to the invention to produce an aerosol suitable for inhalation.

If the formulation according to the invention is nebulized using the method described above (using the RESPIMAT® nebulizer), the quantity delivered should correspond to a defined quantity with a tolerance of not more than 25%, preferably 20% of this amount in at least 97%, preferably at least 98% of all operations of the inhaler (spray actuations). Preferably, between 5 mg and 30 mg of formulation, most preferably between 5 mg and 20 mg of formulation are delivered as a defined mass on each actuation.

However, the formulation according to the invention may also be nebulized by means of inhalers other than those described above, e.g., jet stream inhalers or other stationary nebulizers.

Accordingly, in a further aspect, the invention relates to pharmaceutical formulations in the form of propellant-free inhalable solutions or suspensions as described above combined with a device suitable for administering these formulations, preferably in conjunction with the RESPIMAT® nebulizer. Preferably, the invention relates to propellant-free inhalable solutions or suspensions characterized by the combination of active substances 1 and 2 according to the invention in conjunction with the device known by the name RESPIMAT®. In addition, the present invention relates to the above-mentioned devices for inhalation, preferably the RESPIMAT® nebulizer, characterized in that they contain the propellant-free inhalable solutions or suspensions according to the invention as described hereinbefore.

According to the invention, inhalable solutions which contain the active substances 1 and 2 in a single preparation are preferred. The term “single preparation” also includes preparations which contain the two ingredients 1 and 2 in two-chamber cartridges, as disclosed for example in WO 00/23037. Reference is hereby made to this publication in its entirety.

The propellant-free inhalable solutions or suspensions according to the invention may take the form of concentrates or sterile inhalable solutions or suspensions ready for use, as well as the above-mentioned solutions and suspensions designed for use in a RESPIMAT® nebulizer. Formulations ready for use may be produced from the concentrates, for example, by the addition of isotonic saline solutions. Sterile formulations ready for use may be administered using energy-operated fixed or portable nebulizers which produce inhalable aerosols by means of ultrasound or compressed air by the Venturi principle or other principles.

Accordingly, in another aspect, the present invention relates to pharmaceutical compositions in the form of propellant-free inhalable solutions or suspensions as described hereinbefore which take the form of concentrates or sterile formulations ready for use, combined with a device suitable for administering these solutions, characterized in that the device is an energy-operated free-standing or portable nebulizer which produces inhalable aerosols by means of ultrasound or compressed air by the Venturi principle or other methods.

The Examples which follow serve to illustrate the present invention in more detail without restricting the scope of the invention to the following embodiments by way of example.

### Examples of Formulations

A. Inhalable Powders

| | |
|---|---|
| Ingredient | μg per capsule |

1. Inhalable Powder Formulation

| | |
|---|---|
| oxitropium bromide | 100 |
| formoterol fumarate dihydrate | 12 |
| lactose | 25788 |
| TOTAL | 25000 |

2. Inhalable Powder Formulation

| | |
|---|---|
| oxitropium bromide | 50 |
| salmeterol xinafoate | 50 |
| lactose | 12400 |
| TOTAL | 12500 |

3. Inhalable Powder Formulation

| | |
|---|---|
| oxitropium bromide | 100 |
| salmeterol xinafoate | 50 |
| lactose | 12350 |
| TOTAL | 12500 |

B. Inhalable Aerosols Containing Propellant Gases

| | |
|---|---|
| Ingredient | wt. % |

1. Suspension Aerosol Formulation

| | |
|---|---|
| oxitropium bromide | 0.018 |
| salmeterol × ½ H₂SO₄ | 0.066 |
| soya lecithin | 0.2 |
| TG 11:TG 12 = 2:3 | ad 100 |

2. Suspension Aerosol Formulation

| | |
|---|---|
| oxitropium bromide | 0.039 |
| salmeterol xinafoate | 0.033 |
| absolute ethanol | 0.5 |
| isopropyl myristate | 0.1 |
| TG 227 | ad 100 |

3. Solution Aerosol Formulation

| | |
|---|---|
| oxitropium bromide | 0.055 |
| salmeterol × ½ H₂SO₄ | 0.047 |
| absolute ethanol | 30 |
| purified water | 1.5 |
| anhydrous citric acid | 0.002 |
| TG 134a | ad 100 |

C. Propellant-free Inhalable Solutions

1. Solution for Use with the RESPIMAT ® Nebulizer

| Ingredient | mg/100 mL |
|---|---|
| oxitropium bromide | 1485 |
| salmeterol × ½ H₂SO₄ | 276.7 |
| benzalkonium chloride | 10 |
| sodium edetate | 10 |
| hydrochloric acid (aq) | ad pH 2.9 |
| water | ad 100 mL |

Using the solution in the RESPIMAT® nebulizer leads to a dosage of about 100 μg per dose of 1 and 25 μg per dose of 2.

2. Solution for Use with the RESPIMAT ® Nebulizer

| Ingredient | mg/100 mL |
|---|---|
| oxitropium bromide | 2972.2 |
| salmeterol × ½ H₂SO₄ | 276.7 |
| benzalkonium chloride | 10 |
| hydrochloric acid (aq) | ad pH 2.9 |
| water | ad 100 mL |

Using the solution in the RESPIMAT® nebulizer leads to a dosage of about 200 μg per dose of 1 and 25 μg per dose of 2.

3. Solution for Use with the RESPIMAT ® Nebulizer

| Ingredient | mg/100 mL |
|---|---|
| oxitropium bromide | 1486.1 |
| salmeterol × ½ H₂SO₄ | 553.4 |
| benzalkonium chloride | 10 |
| hydrochloric acid (aq) | ad pH 2.9 |
| water | ad 100 mL |

Using the solution in the RESPIMAT® nebulizer leads to a dosage of about 100 μg per dose of 1 and 50 μg per dose of 2.

4. Solution for Use with the RESPIMAT ® Nebulizer

| Ingredient | mg/100 mL |
|---|---|
| oxitropium bromide | 1486.1 |
| salmeterol × ½ H₂SO₄ | 1106.3 |
| benzalkonium chloride | 8 |
| sodium edetate | 50 |
| hydrochloric acid (aq) | ad pH 2.5 |
| water | ad 100 mL |

Using the solution in the RESPIMAT® nebulizer leads to a dosage of about 100 μg per dose of 1 and 100 μg per dose of 2.

We claim:

1. A pharmaceutical composition comprising:
(a) an oxitropium salt; and
(b) a betamimetic.

2. The pharmaceutical composition according to claim 1, wherein the oxitropium salt is a salt formed with HBr, HCl, HI, monomethylsulfuric acid ester, methanesulfonic acid, methylsulfate, or p-toluenesulfonic acid.

3. The pharmaceutical composition according to claim 1, wherein the betamimetic is a salt formed with hydrochloric acid, hydrobromic acid, sulfuric acid, phosphoric acid, methanesulfonic acid, acetic acid, fumaric acid, succinic acid, lactic acid, citric acid, tartaric acid, 1-hydroxy-2-naphthalenecarboxylic acid, or maleic acid.

4. The pharmaceutical composition according to claim 1, wherein the betamimetic is a salt of salmeterol or formoterol.

5. The pharmaceutical composition according to claim 1, wherein the betamimetic is selected from salmeterol hydrochloride, a salmeterol sulfate, or salmeterol xinafoate.

6. The pharmaceutical composition according to claim 4, wherein the weight ratio of the oxitropium salt to the betamimetic is in a range from about 1:30 to 400:1.

7. The pharmaceutical composition according to claim 1, wherein the betamimetic is selected from formoterol hydrochloride, formoterol sulfate, or formoterol fumarate.

8. The pharmaceutical composition according to claim 1, wherein the weight ratio of the oxitropium salt to the betamimetic is in a range from about 1:5 to 300:1.

9. The pharmaceutical composition according to claim 1, wherein the pharmaceutical composition is in a form suitable for inhalation.

10. The pharmaceutical composition according to claim 1, wherein the pharmaceutical composition is an inhalable powder, a propellant-containing metering aerosol, or a propellant-free inhalable solution or suspension.

11. The pharmaceutical composition according to claim 9, wherein the pharmaceutical composition further comprises a suitable physiologically acceptable excipient selected from the group consisting of: monosaccharides, disaccharides, oligo- and polysaccharides, polyalcohols, and salts.

12. The pharmaceutical composition according to claim 10, wherein the pharmaceutical composition further comprises a suitable physiologically acceptable excipient selected from the group consisting of: monosaccharides, disaccharides, oligo- and polysaccharides, polyalcohols, and salts.

13. The pharmaceutical composition of claim 11, wherein the excipient has a maximum average particle size of up to 250 μm.

14. The pharmaceutical composition of claim 12, wherein the excipient has a maximum average particle size of up to 250 μm.

15. The pharmaceutical composition of claim 13, wherein the excipient has a maximum average particle size of between 10 μm and 150 μm.

16. The pharmaceutical composition of claim 14, wherein the excipient has a maximum average particle size of between 10 μm and 150 μm.

17. A capsule containing a pharmaceutical composition according to claim 1 in the form of an inhalable powder.

18. A capsule containing a pharmaceutical composition according to claim 2 in the form of an inhalable powder.

19. A capsule containing a pharmaceutical composition according to claim 3 in the form of an inhalable powder.

20. A capsule containing a pharmaceutical composition according to claim 4 in the form of an inhalable powder.

21. A capsule containing a pharmaceutical composition according to claim 5 in the form of an inhalable powder.

22. A capsule containing a pharmaceutical composition according to claim 6 in the form of an inhalable powder.

23. A capsule containing a pharmaceutical composition according to claim 7 in the form of an inhalable powder.

24. A capsule containing a pharmaceutical composition according to claim 8 in the form of an inhalable powder.

25. A pharmaceutical composition consisting essentially of:
(a) an oxitropium salt; and
(b) a betamimetic,
wherein the pharmaceutical composition is in the form of an inhalable powder.

26. A pharmaceutical composition according to claim 1, wherein the pharmaceutical composition is a propellant-containing inhalable aerosol and the oxitropium salt and the betamimetic are in dissolved or dispersed form.

27. The pharmaceutical composition according to claim 26, wherein the propellant-containing inhalable aerosol comprises a propellant gas selected from hydrocarbons and halohydrocarbons.

28. The pharmaceutical composition according to claim 26, wherein the propellant-containing inhalable aerosol comprises a propellant gas selected from the group consisting of: n-propane; n-butane; isobutane; and chlorinated and/or fluorinated derivatives of methane, ethane, propane, butane, cyclopropane, and cyclobutane.

29. The pharmaceutical composition according to claim 26, wherein the propellant gas is TG134a, TG227, or a mixture thereof.

30. The pharmaceutical composition according to claim 26, further comprising at least one of a cosolvent, stabilizer, surfactant, antioxidant, lubricant, or means for adjusting the pH of the composition.

31. The pharmaceutical composition according to claim 29, further comprising at least one of a cosolvent, stabilizer, surfactant, antioxidant, lubricant, or means for adjusting the pH of the composition.

32. The pharmaceutical composition according to claim 30, further comprising at least one of a cosolvent, stabilizer, surfactant, antioxidant, lubricant, or means for adjusting the pH of the composition.

33. The pharmaceutical composition according to claim 31, further comprising at least one of a cosolvent, stabilizer, surfactant, antioxidant, lubricant, or means for adjusting the pH of the composition.

34. The pharmaceutical composition according to claim 1, wherein the amount of the oxitropium salt and the betamimetic is up to 5 wt. % of the pharmaceutical composition.

35. A pharmaceutical composition according to claim 1, wherein the pharmaceutical composition is propellant-free inhalable solution or suspension that further comprises a solvent selected from water, ethanol, or a mixture of water and ethanol.

36. The pharmaceutical composition according to claim 35, wherein the pH is between 2 and 7.

37. The pharmaceutical composition according to claim 36, wherein the pH is between 2 and 5.

38. The pharmaceutical composition according to claim 35, wherein the pH of the pharmaceutical composition is adjusted by means of one or more acids selected from the group consisting of: hydrochloric acid, hydrobromic acid, nitric acid, sulfuric acid, ascorbic acid, citric acid, malic acid, tartaric acid, maleic acid, succinic acid, fumaric acid, acetic acid, formic acid, and propionic acid.

39. The pharmaceutical composition according to claim 35, further comprising other co-solvents or excipients.

40. The pharmaceutical composition according to claim 38, further comprising other co-solvents or excipients.

41. The pharmaceutical composition according to claim 39, wherein the co-solvent is selected from the group consisting of alcohols, glycols, polyoxyethylene alcohols, and polyoxyethylene fatty acid esters.

42. The pharmaceutical composition according to claim 39, wherein the co-solvent is selected from the group consisting of: isopropyl alcohol, propylene glycol, polyethylene glycol, polypropylene glycol, glycol ether, and glycerol.

43. The pharmaceutical composition according to claim 39, wherein the excipient is selected from the group consisting of: surfactants, stabilizers, complexing agents, antioxidants, preservatives, flavorings, pharmacologically acceptable salts, and vitamins.

44. The pharmaceutical composition according to claim 43, wherein the excipient is selected from the group consisting of: edetic acid, a salt of edetic acid, ascorbic acid, vitamin A, vitamin E, tocopherols, cetyl pyridinium chloride, benzalkonium chloride, benzoic acid, and benzoate salts.

45. A method of treating inflammatory or obstructive diseases of the respiratory tract in a patient in need of such treatment, the method comprising administering to the patient a therapeutically effective amount of the pharmaceutical composition according to one of claims 1 to 12.

46. The method according to claim 45, wherein the pharmaceutical composition is administered to the patient by inhalation after nebulizing the pharmaceutical composition into an inhalable aerosol using an energy-operated free-standing or portable nebulizer that produces inhalable aerosols by means of ultrasound or compressed air.

47. A pharmaceutical composition consisting essentially of:
(a) an oxitropium salt;
(b) a betamimetic;
(c) a solvent;
(d) benzalkonium chloride; and
(e) sodium edetate.

48. A pharmaceutical composition consisting essentially of:
(a) an oxitropium salt;
(b) a betamimetic;
(c) a solvent; and
(d) benzalkonium chloride.

49. A kit comprising one or more unit dosage containers containing a pharmaceutical composition, each unit dosage container containing a pharmaceutical composition comprising:
(a) an oxitropium salt; and
(b) a betamimetic,
each optionally together with a pharmaceutically acceptable excipient.

50. The kit according to claim 49, further comprising instructions with directions for using the kit.

51. The kit according to claim 49, wherein the betamimetic is a salt of salmeterol or formoterol.

52. The kit according to claim 49, wherein the betamimetic is selected from salmeterol hydrochloride, a salmeterol sulfate, or salmeterol xinafoate.

53. The kit according to claim 49, wherein the betamimetic is selected from formoterol hydrochloride, formoterol sulfate, or formoterol fumarate.

54. A kit comprising:
(a) a first container containing a first pharmaceutical formulation comprising an oxitropium salt; and
(b) a second container containing a second pharmaceutical formulation comprising a comprising a betamimetic, each container each optionally further containing a pharmaceutically acceptable excipient.

55. The kit according to claim 54, further comprising instructions with directions for using the kit.

56. The kit according to claim 54, wherein the betamimetic is a salt of salmeterol or formoterol.

57. The kit according to claim 54, wherein the betamimetic is selected from salmeterol hydrochloride, a salmeterol sulfate, or salmeterol xinafoate.

58. The kit according to claim 54, wherein the betamimetic is selected from formoterol hydrochloride, formoterol sulfate, or formoterol fumarate.
